(19) United States
(12) Patent Application Publication
(10) Pub. No.: US 2018/0207351 A1
(43) Pub. Date: Jul. 26, 2018

(54) Rectal Catheter Plastic Component for a Rectal Catheter Assembly, and Method for Producing a Rectal Catheter Plastic Component

(71) Applicant: Raumedic AG, Munchberg (DE)

(72) Inventor: Björn Gläsel, Kirchenlamitz (DE)

(21) Appl. No.: 15/878,487

(22) Filed: Jan. 24, 2018

(30) Foreign Application Priority Data
Jan. 24, 2017 (DE) 10 2017 201 065.7

(51) Int. Cl. A61M 3/02; A61M 25/10

(52) U.S. Cl. CPC A61M 3/0295; A61M 3/0283; B29C 45/0055; A61M 2210/1064; A61M 25/1036

(57) ABSTRACT

A rectal catheter plastic component is a constituent part of a rectal catheter assembly. The one piece plastic component has a cylindrical main body with an outer jacket wall. A connector component end serves for the attachment of a peripheral hose. A free component end serves for rectal insertion. At least one media lumen, extending along the main body, is arranged between at least one media connector port and at least one media opening. The media connector port is located at the connector component end, and the media opening is configured in the free component end. A further balloon-filling lumen, extending along the main body, extends between a filling connector port and a balloon-filling opening. The filling connector port is located at the connector component end, and the balloon-filling opening is configured in a balloon attachment region of the plastic component for the attachment of a balloon cuff.

### CROSS-REFERENCES TO RELATED APPLICATIONS

This application claims the priority of German Patent Application, Serial No. 10 2017 201 065.7, filed Jan. 24, 2017, pursuant to 35 U.S.C. 119(a)-(d), the content of which is incorporated herein by reference in its entirety as if fully set forth herein.

### FIELD OF THE INVENTION

The invention relates to a rectal catheter plastic component for a rectal catheter assembly. The invention further relates to a method for producing such a rectal catheter plastic component and such a rectal catheter assembly.

### BACKGROUND OF THE INVENTION

Rectal catheter assemblies are known commercially, for example from DE 44 36 796 A1.

### SUMMARY OF THE INVENTION

It is an object of the present invention to design a rectal catheter assembly such that it is more cost-effective.

According to the invention, this object is achieved by a rectal catheter plastic component Rectal catheter plastic component for a rectal catheter assembly,
with a cylindrical main body with an outer jacket wall,
with a connector component end for the attachment of a peripheral hose for the supply and/or withdrawal of media,
with a free component end for rectal insertion,
with at least one media lumen extending along the main body between at least one media connector port is located at the connector component end, and at least one media opening is located in the free component end,
with at least one further balloon-filling lumen extending along the main body between a filling connector port is located at the connector component end, and at least one balloon-filling opening is located in a balloon attachment region of the plastic component for the attachment of a balloon cuff,
wherein the rectal catheter plastic component is configured in one piece, and
wherein the rectal catheter plastic component is made of a thermoplastic material.

According to the an implementation of the invention, it was found that a cylindrical main body having the necessary lumina, on the one hand, and a connector component end for the attachment of a peripheral hose, on the other hand, can be configured as a one-piece plastic component. This avoids a complicated multi-part design in which the various component parts would have to be sealed off from each other and assembled with some difficulty. Problems relating to hygiene and sterilization are also eliminated. The main body is designed as a multi-lumen body and can have exactly two lumina or also more than two lumina, for example three lumina, four lumina, five lumina or still more lumina

With the rectal catheter plastic component made of thermoplastic material, it is possible to manufacture the rectal catheter plastic component and/or subcomponents thereof by at least one of the following manufacturing process types: extrusion, injection molding or blow molding.

A lumen configuration in which adjacent lumina are separated from each other by a web extending along the main body, leads to a simple cross-sectional structure of the main body. The web is formed integrally in the main body.

A lumen configuration, in which they have a convex cross section, can avoid edges extending along the lumina. The convex cross sections of the lumina can be circular, elliptic or oval.

A design of the rectal catheter plastic component as an injection-moulded component permits mass production.

An annular stop formed integrally on the jacket wall of the main body permits fixing of the rectal catheter plastic component.

A further catheter lumen extending along the main body and provided for the insertion of a further intestinal catheter widens the possible range of uses of the rectal catheter assembly equipped with such a rectal catheter plastic component.

A production method for producing a rectal catheter plastic component according to an embodiment of the invention includes the following steps:
injection moulding of a preform having the main body and at least two lumina extending therein,
closing an axial end of at least one of the lumina, which is used as the balloon-filling lumen, with a closure plug, and
punching the at least one balloon-filling opening into the outer jacket wall of the main body.

The rectal catheter plastic component can be constructed from a relatively simple preform, which can be produced in particular by injection moulding. The preform can have an already integrally formed connector component end. The production method can include the method step of shaping the free component end. Here, the free component end can be configured in particular with a convex tip, such that the rectal insertion of the free component end is made easier.

A media opening can be formed via a tube opening of the preform. Alternatively, a media opening can be formed by punching the at least one media opening into the free component end.

The advantages of a rectal catheter assembly, comprising a thermal shaping process and/or comprising a material-removing process for forming a free component end from a free tube end of the preform, correspond to those that have already been explained above with reference to the rectal catheter plastic component. The balloon cuff can be adhesively bonded and/or welded to the rectal catheter plastic component.

A rectal catheter assembly with a rectal catheter plastic component according to an embodiment of the invention includes a balloon cuff which is fixed in the balloon attachment region on the rectal catheter plastic component, and a peripheral hose for the supply and/or withdrawal of media, can be marketed as a complete product.

A separate connector component between the rectal catheter plastic component and the peripheral hose simplifies the design of a connector component end on the rectal catheter plastic component.

Illustrative embodiments of the invention are explained in more detail below with reference to the drawings.

### BRIEF DESCRIPTION OF THE DRAWINGS

FIGS. 1 to 6 show axial sections through a plastic component during its processing in the production of a rectal catheter plastic component;

FIGS. 7 and 8 show two alternative embodiments of a cross section of a cylindrical main body of the plastic component, in each case with two lumina extending along the main body;

FIG. 9 shows a perspective view of a rectal catheter assembly with a further embodiment of a rectal catheter plastic component which, except for the design of a connector component end, corresponds to the embodiment of the rectal catheter plastic component according to FIG. 6;

FIG. 10 shows a side view of the rectal catheter assembly according to FIG. 9;

FIG. 11 shows a view of the rectal catheter assembly seen from the viewing direction XI in FIG. 10;

FIG. 12 shows an axial section through the rectal catheter assembly according to FIGS. 9 to 11;

FIG. 13 shows a perspective view of a further embodiment of a rectal catheter assembly, with a further embodiment of a rectal catheter plastic component;

FIG. 14 shows a side view of the rectal catheter assembly according to FIG. 13;

FIG. 15 shows a front view of the rectal catheter assembly, seen from the viewing direction XV in FIG. 14;

FIG. 16 shows an axial section through the rectal catheter assembly according to FIGS. 13 to 15; and

FIG. 17 shows a perspective view of a portion of a multi-lumen peripheral hose, suitable for attachment to the rectal catheter assembly according to FIGS. 13 to 16.

### DETAILED DESCRIPTION OF THE PREFERRED EMBODIMENTS

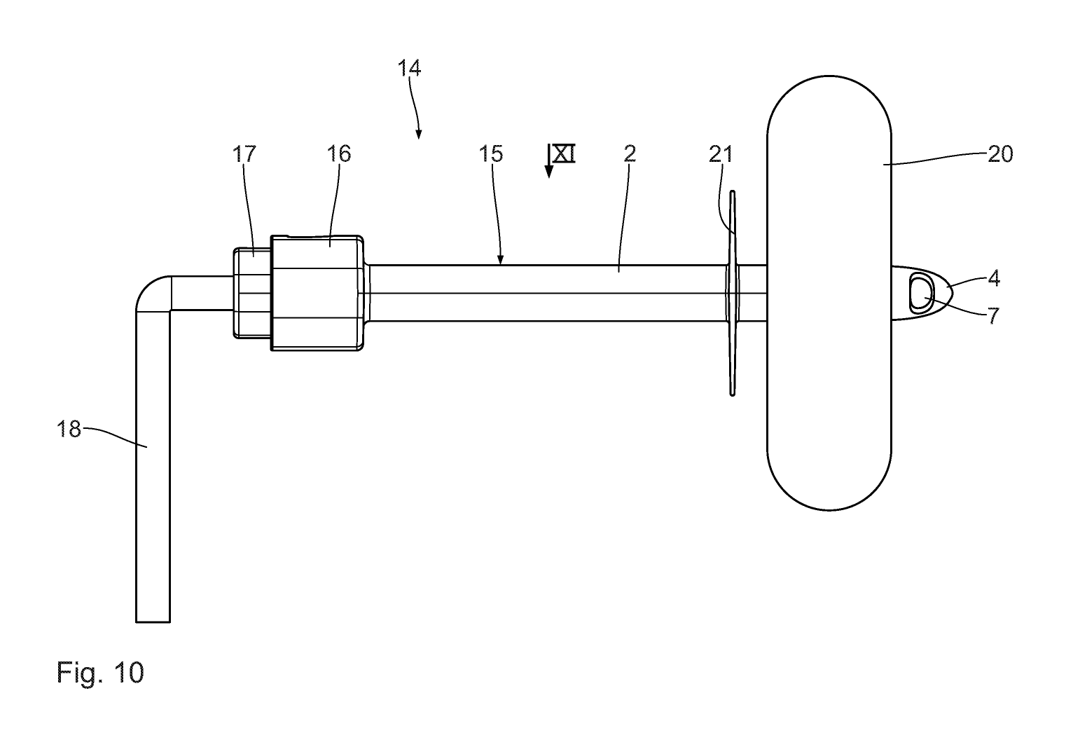

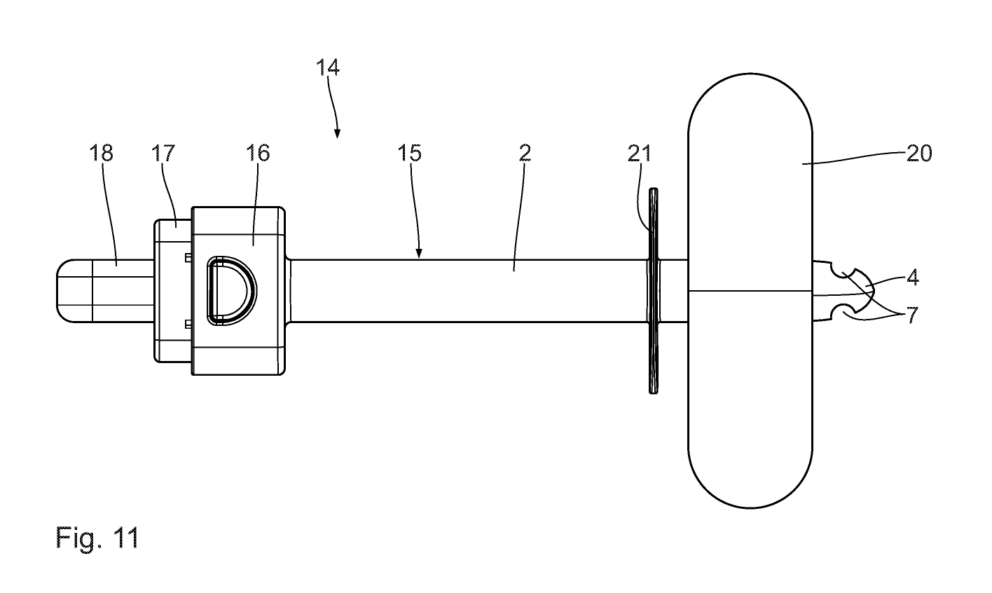

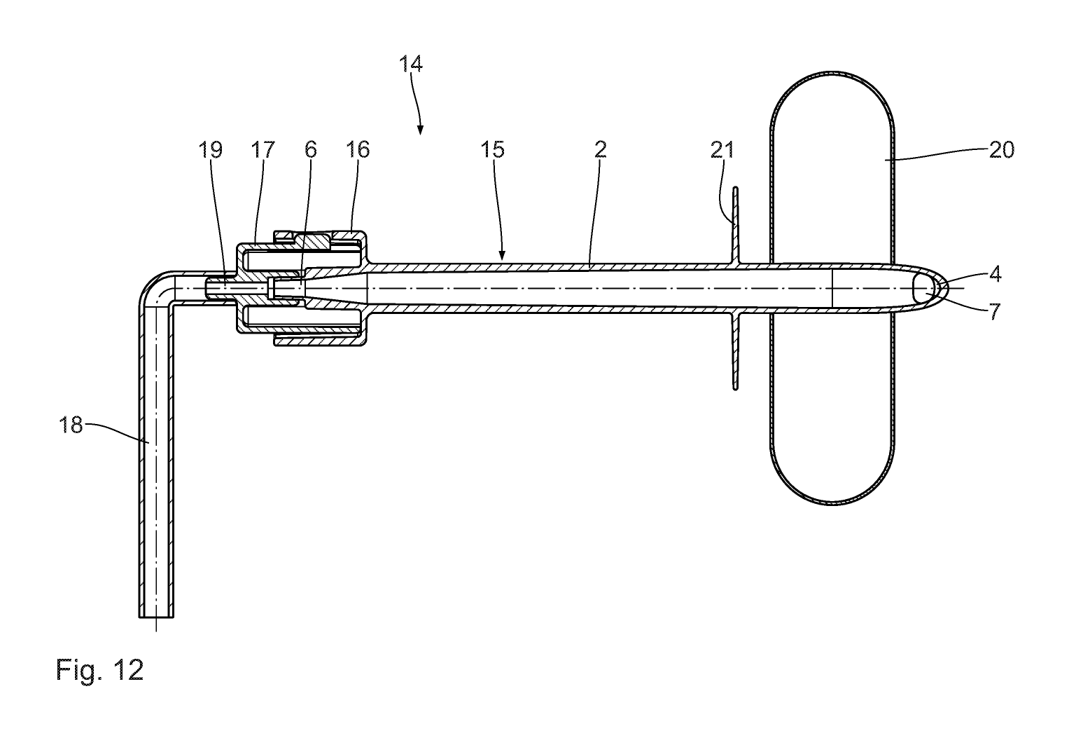

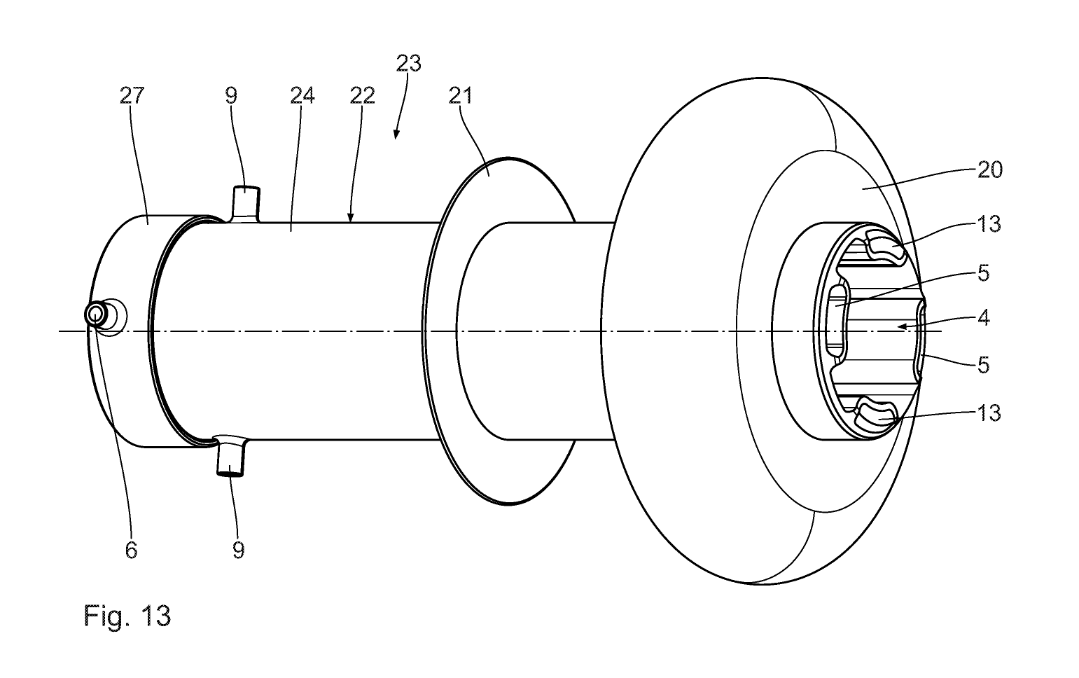

A rectal catheter plastic component 1 (cf. FIG. 6) is a constituent part of a rectal catheter assembly, for which embodiments are also described below in connection with FIGS. 9 to 17. Depending on the embodiment of the rectal catheter assembly, the latter can be used on or in a patient for a period of minutes, hours, days or weeks.

The rectal catheter plastic component 1 is configured as a one-piece injection-moulded component. The rectal catheter plastic component 1 is made of a thermoplastic material. A Shore hardness of the material can be in the range between 80A and 90A. Such a material is also designated as semi-rigid thermoplastic material. The rectal catheter plastic component 1 can be produced from polyurethane

The plastic component 1 has a cylindrical main body 2 with an outer jacket wall. A connector component end 3 of the plastic component 1, which is designed as an attachment flange in the embodiment according to FIG. 6, serves for the attachment of a peripheral hose (cf. the embodiments according to FIGS. 9 to 17) for the supply and/or withdrawal of media. The entire rectal catheter assembly can be used for administering an enema and/or for single or continuous bowel cleansing.

Moreover, the plastic component 1 has a free component end 4 for rectal insertion through the anus into the intestine of a patient.

At least one media lumen, in the illustrative embodiment according to FIG. 6 exactly one media lumen 5, extends along the main body 2 between at least one media connector port 6 for attachment of the peripheral hose, configured at the connector component end 3, and at least one media opening, four media openings 7 in the embodiment according to FIG. 6, configured in the free component end 4. The free component end 4 is designed as a convex, approximately dome-shaped, hollow termination of the cylindrical main body 2. The media openings 7 are configured radially in the jacket wall of the main body 2.

Moreover, at least one further balloon-filling lumen, in the plastic component 1 exactly one balloon-filling lumen 8, extends along the main body 2. The balloon-filling lumen 8 extends between a filling connector port 9, which is likewise configured at the connector component end 3, and at least one balloon-filling opening, exactly one balloon-filling opening 10 in the plastic component 1 according to FIG. 6. The balloon-filling opening is configured in an axial balloon attachment region of the plastic component 1 for attachment to a balloon cuff, which is described below also in connection with the embodiments according to FIGS. 9 to 17. The balloon-filling opening 10 is configured radially in the outer jacket wall of the cylindrical main body 2.

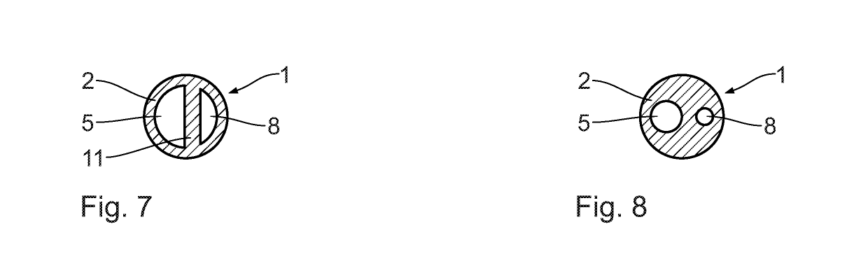

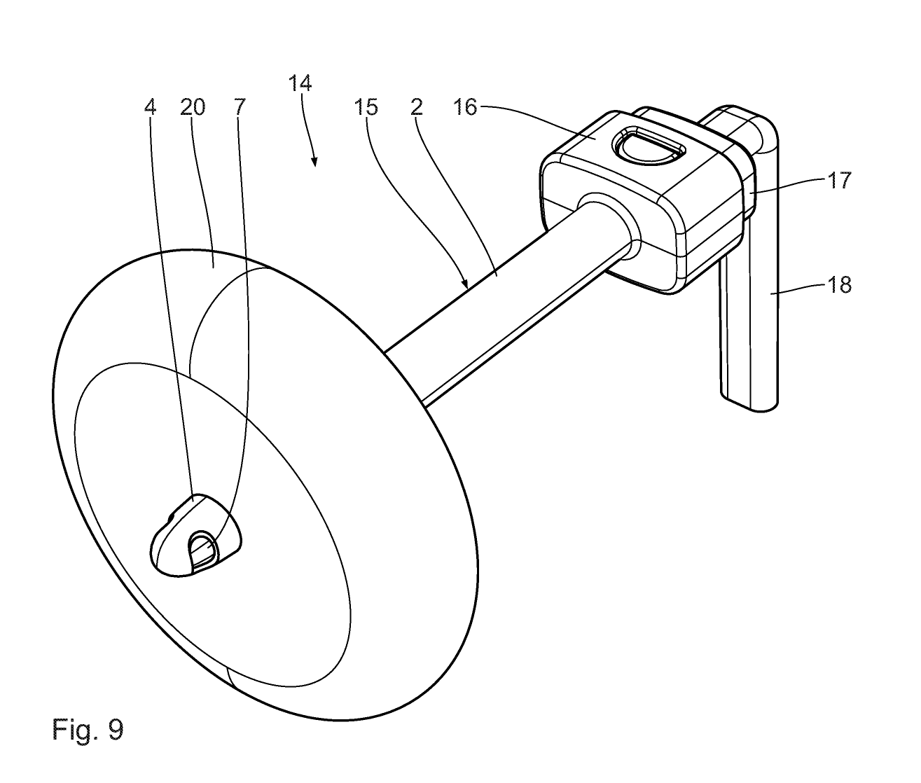

FIGS. 7 and 8 show two examples of the cross-sectional configuration of the media lumen 5, on the one hand, and of the balloon-filling lumen 8, on the other hand.

In the embodiment according to FIG. 7, the two lumina 5, 8 are separated from each other by a web 11 extending along the main body 2. The web 11 is formed integrally in the main body 2.

In the embodiment according to FIG. 8, the two lumina 5, 8 each have a convex cross section, namely a circular or an oval cross section.

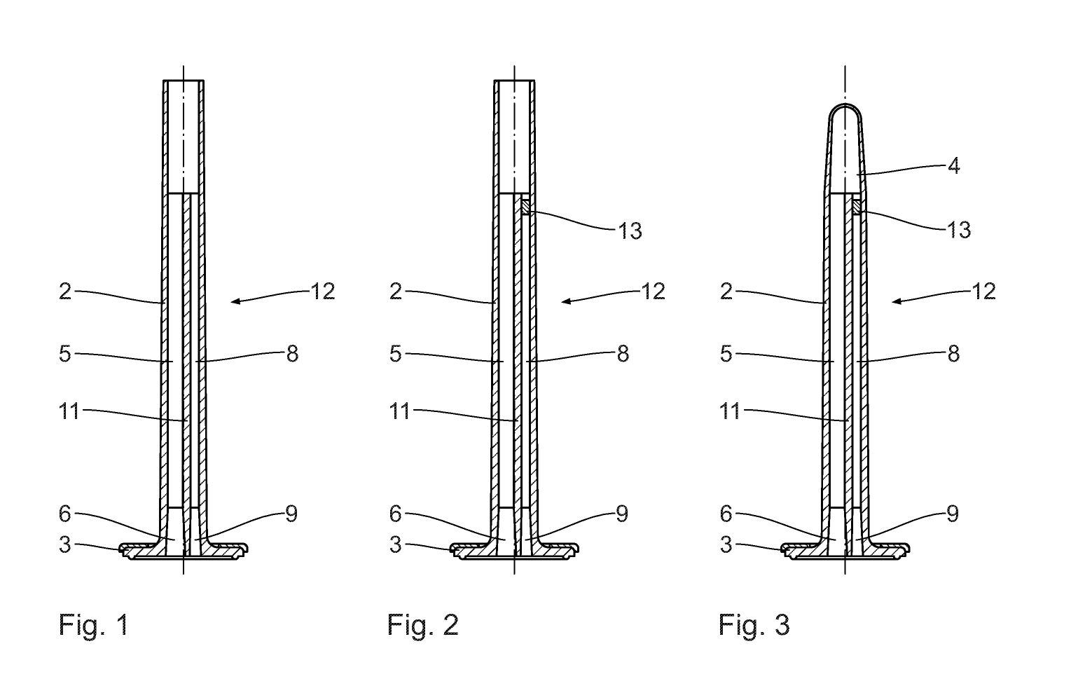

FIGS. 1 to 6 show stages during the production of the rectal catheter plastic component 1.

FIG. 1 shows a preform, which is produced by injection moulding and which has the main body 2, the media lumen 5, the balloon-filling lumen 8 and the connector component end 3. A free end of this preform 12 is designed as an open tube end into which the two lumina 5, 8 open.

FIG. 2 shows the result of a subsequent production step in which an axial end of the balloon-filling lumen 8 is closed with a closure plug 13. The closure plug 13 can be an adhesive stopper.

FIG. 3 shows the result of a subsequent production step in which the free component end 4 is formed from the free tube end of the preform 12 by a thermal shaping process and/or by a material-removing process.

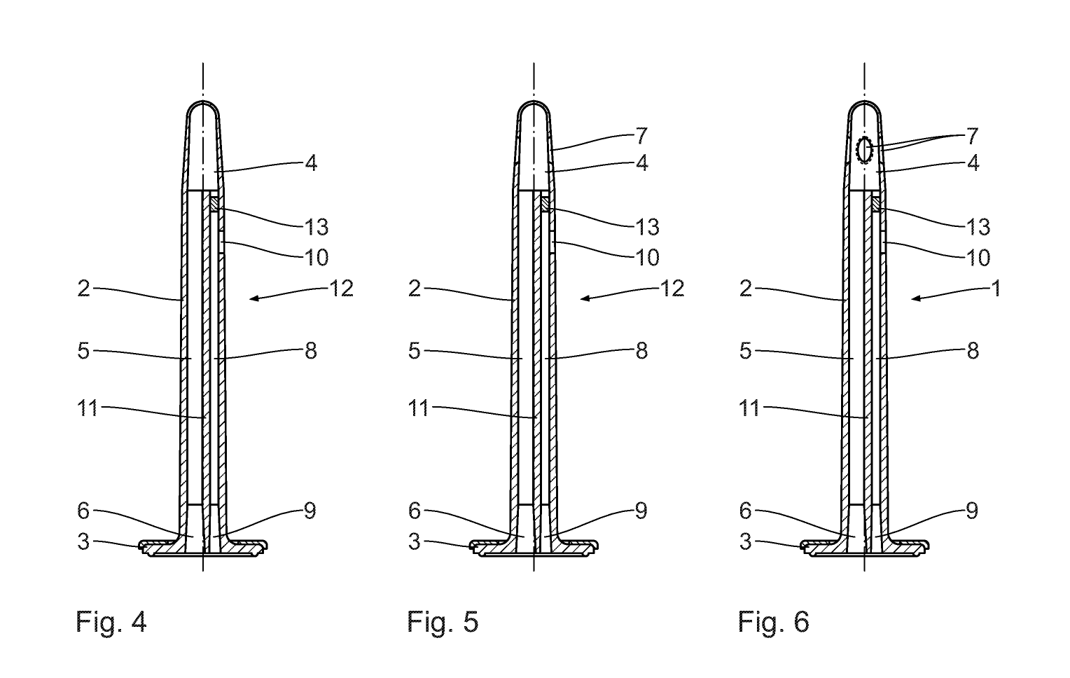

FIG. 4 shows the result of a subsequent production step in which the balloon-filling opening 10 is punched into the outer jacket wall of the main body 2.

FIGS. 5 and 6 show the results of subsequent steps for producing the plastic component 1, in which steps the media openings 7 are punched into the free component end 4.

A further embodiment of a rectal catheter plastic component 15 as a constituent part of a rectal catheter assembly 14 is explained below with reference to FIGS. 9 to 12. Component parts corresponding to those already explained above with reference to FIGS. 1 to 8 carry the same reference numbers and are not discussed again in detail.

A rectal catheter plastic component 15 differs from the plastic component 1 in terms of the design of a connector component end 16. The latter is designed as a connector bushing into which a further, separate connector component 17 of the rectal catheter assembly 14 latches. The rectal catheter assembly 14 thus has a two-part connector for connecting the rectal catheter plastic component 15 to a peripheral hose 18 for the supply and/or withdrawal of media. The peripheral hose 18 is configured as a multi-lumen hose on the one hand for supplying water for the balloon filling and, on the other hand, for the supply and withdrawal of media. The media connector port 6 of the connector component end 16 is fludically connected to an inner lumen of the peripheral hose 18 via a connector lumen 19 of the separate connector component 17.

The rectal catheter assembly 14 moreover comprises a balloon cuff 20 which is fixed to the plastic component 15 in the attachment region thereof The cuff 20 can be adhesively bonded and/or welded to the outer jacket wall of the main body 2. During the use of the rectal catheter assembly 14, the balloon cuff 20 can be filled with water at body temperature. The balloon cuff 20 can be produced from polyurethane

The rectal catheter plastic component 15 moreover has an annular stop 21 which is integrally formed on the outer jacket wall of the main body 2. The annular stop 21 lies at an axial distance from the balloon cuff 20, such that the latter is arranged between the annular stop 21 and the free component end 4, where the attachment region of the plastic component 15 also lies. The annular stop 21 interacts with the balloon cuff 20 to fix the rectal catheter assembly 14 in the intestine.

During the use of the rectal catheter assembly 14, the latter is inserted, with the balloon cuff 20 initially empty, through the anus and into the intestine of the patient, until the sphincter of the intestine lies axially between the balloon cuff 20 and the annular stop 21. Water at body temperature is then introduced into the balloon cuff 20 via the balloon-filling lumen 8, and the rectal catheter assembly 14 is thus fixed in the intestine. Supply and/or withdrawal of media between the periphery and the intestine can now take place via the media lumen 5. The radial arrangement of the media openings 7 in the jacket wall of the free component end 4 provides for a gentle supply of media.ö

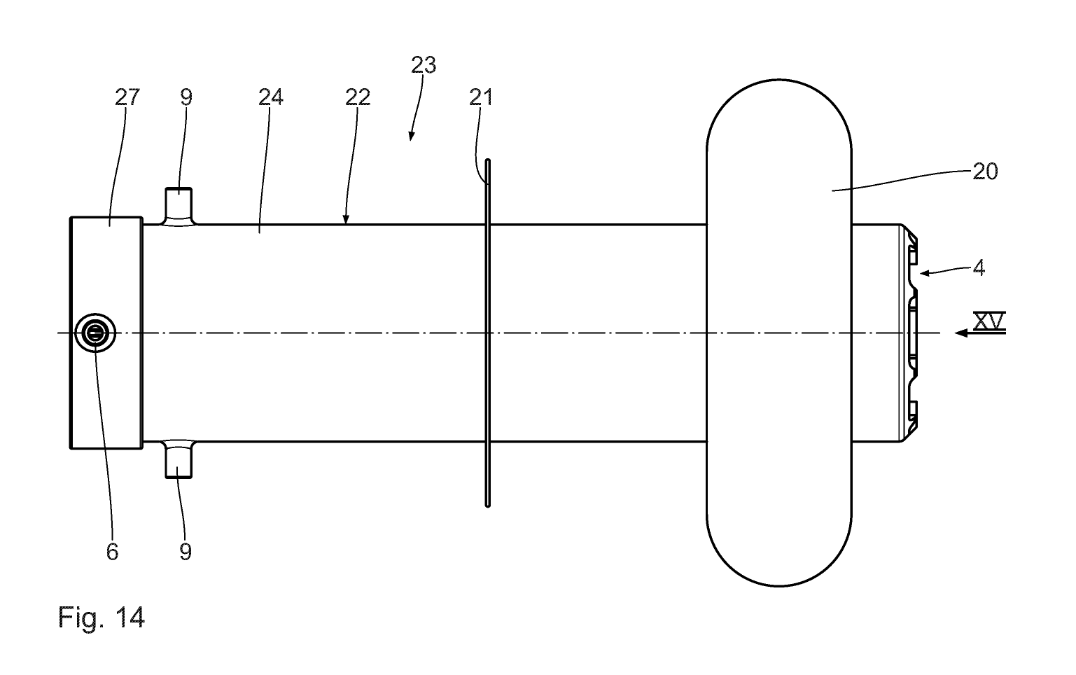

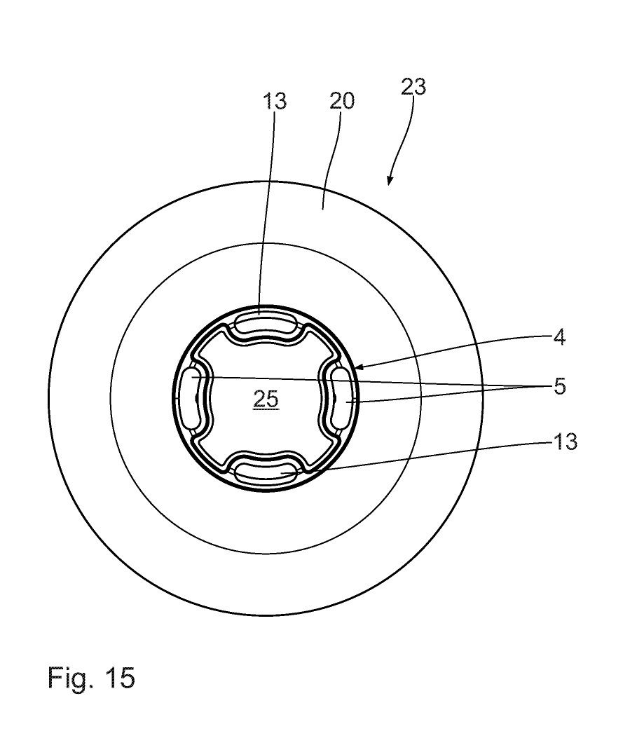

A further embodiment of a rectal catheter plastic component 22 for a further embodiment of a rectal catheter assembly 23 is described below with reference to FIGS. 14 to 17. Component parts corresponding to those already explained above with reference to FIGS. 1 to 12 carry the same reference numbers and are not discussed again in detail.

The rectal assembly 23 serves for more prolonged use and permits the insertion of an additional intestinal catheter. For this purpose, a main body 24 of the rectal catheter plastic component 22 has a catheter lumen 25 (cf. FIG. 15) which extends along the main body 24 and serves for the insertion of the further intestinal catheter. The rectal catheter plastic component 22 has four further lumina, of which two can be used as media lumina 5 and two as balloon-filling lumina 8. The balloon-filling lumina 8 each have a closure plug 13 at their axially free ends.

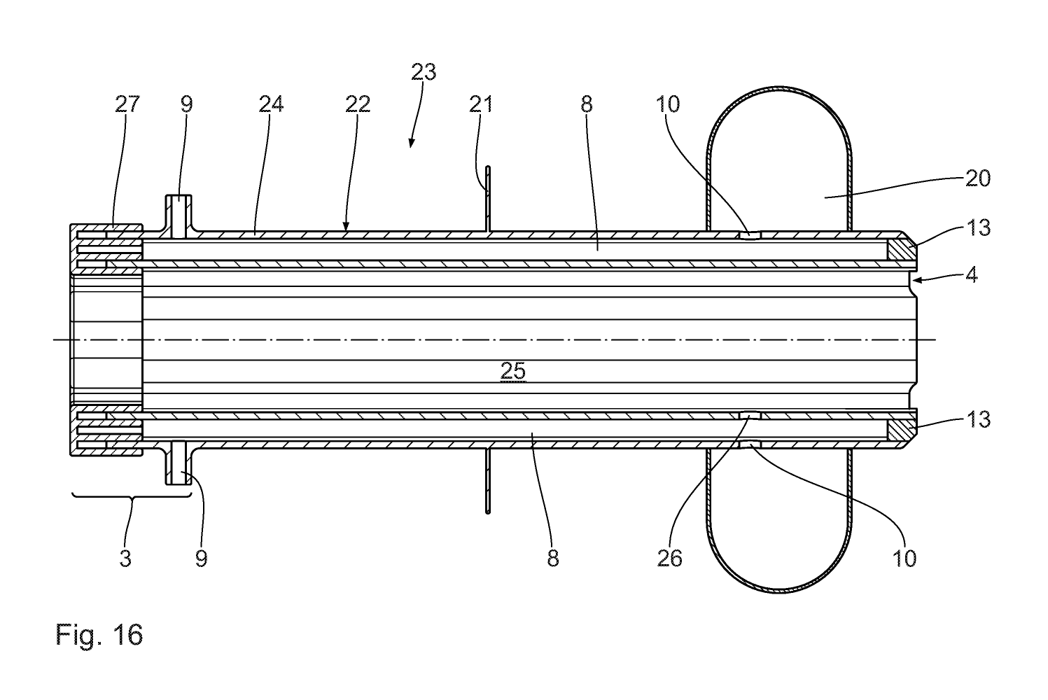

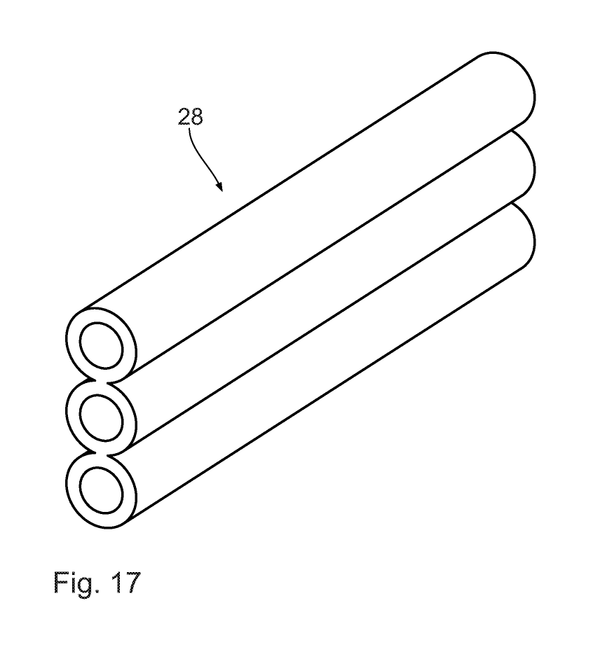

In the rectal catheter plastic component 22, the connector component end 3 is designed as an axial end portion of the main body 24. As is shown in the example of the lower filling lumen 8 in FIG. 16, the latter can also be fluidically connected to the catheter lumen 25 via a punch 26. In the rectal catheter assembly 23, a media connector port 6 is configured in an end cap 27 that closes the main body 24 at the end opposite the free component end 4. Further ports 9, which can be used as filling connector ports for example, can be integrally formed directly on the outer jacket wall of the main body 24. The end cap 27 has a central through-opening which is flush with the catheter lumen 25 of the rectal catheter plastic component 22.

FIG. 17 shows an embodiment of a peripheral hose 28 for the rectal catheter assembly 23, for example in the form of a hose with three lumina. These three lumina can be attached to the ports 6 and 9 of the rectal catheter assembly 23.

During the use of the rectal catheter assembly 23, an additional intestinal catheter (not shown) can be inserted through the catheter lumen 25 of the rectal catheter plastic component 22 into the intestine of the patient.

What is claimed is:

1. A rectal catheter plastic component for a rectal catheter assembly comprising:
a cylindrical main body with an outer jacket wall;
a connector component end for the attachment of a peripheral hose for at least one of the supply and the withdrawal of media;
a free component end for rectal insertion;
at least one media lumen extending along the main body between at least one media connector port that is located at the connector component end, and at least one media opening that is located in the free component end; and
at least one further balloon-filling lumen extending along the main body between a filling connector port that is located at the connector component end, and at least one balloon-filling opening that is located in a balloon attachment region of the plastic component and that is configured for the attachment of a balloon cuff,
wherein the rectal catheter plastic component is configured in one piece, and
wherein the rectal catheter plastic component is made of a thermoplastic material.

2. The rectal catheter plastic component according to claim 1, wherein the lumina are adjacent to one another and are separated from each other by a web extending along the main body.

3. The rectal catheter plastic component according to claim 1, wherein the lumina have a convex cross section.

4. The rectal catheter plastic component according to claim 1, wherein the component is an injection-moulded component.

5. The rectal catheter plastic component according to claim 1, further comprising an annular stop formed integrally on the jacket wall of the main body.

6. The rectal catheter plastic component according to claim 1, further comprising a catheter lumen extending along the main body and provided for the insertion of a further intestinal catheter.

7. A method for producing a rectal catheter plastic component for a rectal catheter assembly, the rectal catheter plastic component comprising
a cylindrical main body with an outer jacket wall,
a connector component end for the attachment of a peripheral hose for at least one of the supply and the withdrawal of media,
a free component end for rectal insertion,
at least one media lumen extending along the main body between at least one media connector port that is located at the connector component end, and at least one media opening that is located in the free component end (4), and
at least one further balloon-filling lumen extending along the main body between a filling connector port that is located at the connector component end, and at least one balloon-filling opening that is located in a balloon attachment region of the plastic component for the attachment of a balloon cuff,
wherein the rectal catheter plastic component is configured in one piece, and
wherein the rectal catheter plastic component is made of a thermoplastic material,
the method comprising the steps of:
injection moulding a preform having the main body and at least two lumina extending therein;
closing an axial end of at least one of the lumina, which is used as the balloon-filling lumen, with a closure plug; and
punching the at least one balloon-filling opening into the outer jacket wall of the main body.

8. The method according to claim 7, further comprising punching the at least one media opening into the free component end.

9. The method according to claim 7, further comprising forming a free component end from a free tube end of the preform via one of a thermal shaping process and a material-removing process.

10. A rectal catheter assembly comprising:
a rectal catheter plastic component for a rectal catheter assembly, the rectal catheter plastic component including
a cylindrical main body with an outer jacket wall,
a connector component end for the attachment of a peripheral hose for at least one of the supply and the withdrawal of media,
a free component end for rectal insertion,
at least one media lumen extending along the main body between at least one media connector that is located at the connector component end, and at least one media opening that is located in the free component end,
at least one further balloon-filling lumen extending along the main body between a filling connector port that is located at the connector component end, and at least one balloon-filling opening that is located in a balloon attachment region of the plastic component for the attachment of a balloon cuff,
wherein the rectal catheter plastic component is configured in one piece, and
wherein the rectal catheter plastic component is made of a thermoplastic material; and
a balloon cuff which is fixed in the balloon attachment region on the rectal catheter plastic component.

11. The rectal catheter assembly according to claim 10, further comprising a peripheral hose for at least one of the supply and the withdrawal of media.

12. The rectal catheter assembly according to claim 10, further comprising a separate connector component located between the rectal catheter plastic component and the peripheral hose.
